(19) United States
(12) Patent Application Publication
(10) Pub. No.: US 2025/0057584 A1
(43) Pub. Date: Feb. 20, 2025

(54) MULTIFUNCTIONAL ELECTROSURGICAL DEVICE FOR TISSUE DISSECTION AND HEMOSTASIS

(71) Applicant: Medtronic Advanced Energy LLC, Portsmouth, NH (US)

(72) Inventors: Xiaoming CHENG, Marana, AZ (US); Drew WILSON, Boulder, CO (US); Matt S. PALM, Black Hawk, CO (US)

(21) Appl. No.: 18/798,915

(22) Filed: Aug. 9, 2024

Related U.S. Application Data
(60) Provisional application No. 63/520,084, filed on Aug. 16, 2023.

(51) Int. Cl. A61B 18/14; A61B 18/00; A61B 18/12

(52) U.S. Cl. CPC A61B 18/14; A61B 18/1206; A61B 2018/00083; A61B 2018/00178; A61B 2018/00196; A61B 2018/00922; A61B 2018/1253; A61B 2018/126; A61B 2018/1405; A61B 2018/1467; A61B 2218/002; A61B 2218/007

(57) ABSTRACT

A multifunctional electrosurgical device comprising a proximal portion comprising at least one electrical connector coupled to an electrical surgical unit, one or more irrigation tubes coupled to a fluid source and one or more suction tubes coupled to a waste receptacle. A shaft, at a distal portion, comprising a first bipolar electrode extending distally from the distal portion, configured to selectively provide a delivered electrical current to a target treatment area and a second bipolar electrode, extending distally from the distal portion, configured to receive a return electrical current from the target treatment area. The distal portion further comprising a retractable monopolar electrode, selectively extendable and configured to provide a delivered electrical current to the target treatment area and one or more user inputs for control of the bipolar or monopolar electrode activation, monopolar electrode expendability and retractability, irrigation of a fluid and suction at a treatment area.

### CROSS-REFERENCE TO RELATED APPLICATION

This application claims the benefit of and priority to U.S. Provisional Patent Application No. 63/520,084 filed Aug. 16, 2023, the entire disclosure of which is incorporated by reference herein.

### FIELD

The present technology is generally related to instruments or tools used in performing surgery on a patient and, more particularly, to electrosurgical devices, systems and methods that provide for cutting, coagulation, hemostasis or sealing of body tissues, including bone, with an electrosurgical device.

### BACKGROUND

This disclosure relates generally to the field of medical devices, systems and methods for use in surgical procedures. More specifically, this disclosure relates to electrosurgical devices, systems and methods that provide for cutting, coagulation, dissection, hemostasis, or sealing of bodily tissues, including bone, with an electrosurgical device. Such electrosurgical devices can be used for, but not limited to orthopedic, spine, thoracic, or open abdominal surgery.

An electrosurgical device may include a handheld unit having a distal end with one or more electrodes. The one or more electrodes can be positioned proximate the target tissue such that an electrical current is introduced into the tissue. The resulting generated heat can be used to cut, coagulate, or induce metabolic processes in the target tissue. The electrosurgical device can be used with an electrosurgical generator which generally provides power and electrical energy in the form of radio frequency (“RF”) energy via either of two handpiece topologies (or a particular combination thereof): monopolar or bipolar.

During monopolar operation, an active electrode introduces current into the target tissue. The current returns through a return electrode separately located on a patient's body. Accordingly, the monopolar handpiece has only one wire for the treatment signal in the monopolar connector—the second contact, known as the “return signal” exists in a different connector known as a “return-pad connector.” During bipolar operation, current is introduced into, and returned from, the target tissue via “active” and “return” electrodes located on the bipolar handpiece.

Conventional electrosurgical devices used for electrosurgical tissue treatment face an array of challenges that can vary across procedures. Some challenges that can arise are the use of multiple different devices to perform individual functions, thereby both complicating the procedure and occupying a greater amount of a limited space, both internal to the patient and within the operating environment. To address these issues, some multi-functional electrosurgical devices capable of performing multiple techniques have been developed.

Despite having the ability to perform different functions with a single device, the device can face an array of challenges. For example, when monopolar function is desired, only one of the electrodes of the device are utilized and the deactivated other electrode(s) may obstruct the view of the surgeon during the monopolar operation. Furthermore, the deactivated electrode(s) may unnecessarily prevent the monopolar electrode from entering smaller spaces or tissue areas that could otherwise be accessed if the unused electrode was not exposed. Further still, devices may not perform similarly to independent bipolar and monopolar devices. These challenges can both complicate the procedure and occupy a greater amount of a limited space, both internal to the patient and within the operating environment.

Thus, a need therefore exists for an improved, multifunctional electrosurgical devices, particularly one that is easier and more efficient to use with improved integration and adaptability.

### SUMMARY

The techniques of this disclosure generally relate to instruments or tools used in performing surgery on a patient and, more particularly, to electrosurgical devices, systems and methods that provide for cutting, coagulation, hemostasis or sealing of body tissues, including bone, with an electrosurgical device.

In one aspect, the present disclosure provides a multifunctional electrosurgical device comprising a proximal portion comprising at least one of an electrical connector coupled to an electrical surgical unit, one or more irrigation tubes coupled to a fluid source and one or more suction tubes coupled to a waste receptacle. An elongated, oval shaft including a distal portion comprising a first bipolar electrode extending distally from the distal portion, the first electrode configured to selectively provide a delivered electrical current to a target treatment area and a second bipolar electrode extending distally from the distal portion, the second electrode configured to receive a return electrical current from the target treatment area, wherein the first and second electrodes are laterally and spatially separated. The first and second bipolar electrodes integrated with the one or more suction tubes. The distal portion further comprising a retractable monopolar electrode, positioned in between the first and second electrode, selectively extendable from the distal portion and configured to selectively provide a delivered electrical current to the target treatment area. Aa handle portion in-between the proximal portion and distal portion, including one or more user input mechanisms for control of at least one of the bipolar or monopolar electrode activation, extraction and retraction of the retractable monopolar electrode, irrigation of a fluid, and suction at the treatment area.

In another aspect, the present disclosure provides a multifunctional electrosurgical device comprising a proximal portion comprising at least one of an electrical connector coupled to an electrical surgical unit and one or more irrigation tubes coupled to a fluid source. A cylindrical shaft including a distal portion comprising a first bipolar electrode extending distally from the distal portion, the first electrode configured to selectively provide a delivered electrical current to a target treatment area and a second bipolar electrode extending distally from the distal portion, the second electrode configured to receive a return electrical current from the target treatment area, wherein the first and second electrodes are curved and contour to the sides of the distal portion of the cylindrical shaft. The first and second bipolar electrodes integrated with the one or more irrigation tubes. The distal portion further comprising a retractable monopolar electrode, positioned in between the first and second electrode, selectively extendable from the distal portion and configured to selectively provide a delivered electrical current to the target treatment area. A handle portion in-between the proximal portion and distal portion, including one or more user input mechanisms for control of at least one of the bipolar or monopolar electrode activation, extraction and retraction of the retractable monopolar electrode, irrigation of a fluid, and suction at the treatment area.

In another aspect, the present disclosure provides a method of performing electrosurgery with a multifunctional electrosurgical device, the method comprising selectively delivering, via activating a first bipolar electrode, a delivered current to a target treatment area. Selectively receiving, via activating a second bipolar electrode, a return electrical current from the target treatment area. Selectively delivering, via activating a retractable monopolar electrode, a delivered current to the treatment area, wherein the retractable monopolar is selectively extruded and retracted from within a distal portion of a shaft of the multifunctional electrosurgical device. Selectively delivering, via at least one irrigation tubing, a fluid to the target treatment area. Selectively suctioning, via at least one suction tubing, remnants and fluid from the target treatment area, wherein one or more user input mechanisms are configured to activate and deactivate each aspect of selectively activating and deactivating the bipolar electrodes, activating and deactivating the monopolar electrode, delivering fluid to the target treatment area and suctioning remnants and fluid from the target treatment area.

Examples of the present disclosure advantageously reduce the number of surgical tools required in the field, allowing irrigation and aspiration of surgical fluid to be performed by the same tool providing the electrosurgery.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a front view on a medical system having an electrosurgical unit, a fluid source, and a multifunctional electrosurgical device, according to embodiments.

FIG. 2 is a perspective view of a multifunctional electrosurgical device, according to embodiments.

FIG. 3 is a perspective view of an example distal portion of a multifunctional electrosurgical device, according to embodiments.

FIG. 4 is a perspective view of an example distal portion of a multifunctional electrosurgical device, according to embodiments.

FIG. 5 is an example block diagram for a method of use of a multifunctional electrosurgical device, according to embodiments.

### DETAILED DESCRIPTION

As described herein, embodiment of the present disclosure provides systems and methods related instruments or tools used in performing surgery on a patient and, more particularly, to electrosurgical devices, systems and methods that provide for cutting, coagulation, hemostasis or scaling of body tissues, including bone, with an electrosurgical device.

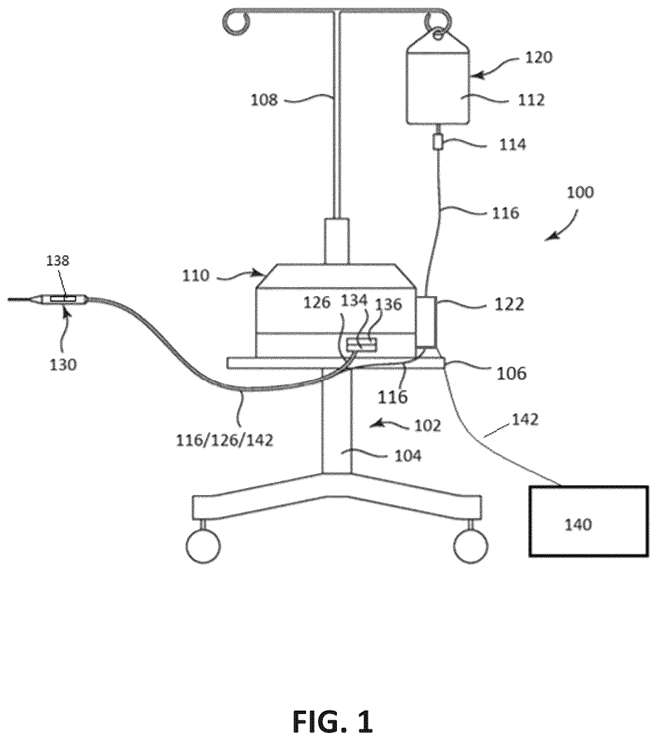

FIG. 1 depicts a front view illustrating an embodiment of a system according to the present disclosure including an example electrosurgical unit in combination with a fluid source, and an example electrosurgical device, according to embodiments. The system 100 may be carried on a movable or portable hand cart 102 having a support structure 104 comprising a hollow (or solid) cylindrical post which includes a platform 106. The platform 106 including a pedestal table to provide a flat, stable surface allowing an electrosurgical unit 110 to rest. The hand cart 102 may include a pole 108 having an adjustable height by sliding the pole 108 up or down. A fluid source 120 may be supported at the top of the pole 108, but not limited thereto.

The fluid source 120 may comprise a bag or other container from which fluid 112 may flow through irrigation tubing 116 and to a multifunctional electrosurgical device 130, which may be dispensable therefrom. In embodiments, a drip chamber 114 may be utilized between the fluid source 120 and the multifunctional electrosurgical device 130. In embodiments, the fluid 112 may include saline and may include physiologic saline such as sodium chloride (NaCl) 0.9% weight/volume solution. Saline, an electrically conductive fluid, and other suitable electrically conductive fluids may be used. In embodiments, the fluid may include a nonconductive fluid, such as deionized water, which may still provide advantages over using no fluid and may support cooling of portions of the multifunctional electrosurgical device 130 and tissue or reducing the occurrence of tissue sticking to the multifunctional electrosurgical device 130.

In embodiments, the irrigation tubing 116 may flow through pump 122 to convey fluid to the multifunctional electrosurgical device 130 and control fluid flow. In embodiments, the pump 122 may be a peristaltic pump, such as a rotary peristaltic pump. With a rotary peristaltic pump, a portion of the delivery tubing 116 is loaded into the pump head by raising and lowering the pump head in a predetermined manner. Fluid 112 may be conveyed within the delivery tubing 116 through the irrigation tubing 116 by way of intermittent forces placed on the external surface of the delivery tubing. Peristaltic pumps are often applied during operation of the multifunctional electrosurgical device 130 because the mechanical elements of the pump places forces on the external surface of the delivery tubing and do not come into direct contact with the fluid 112, which can reduce the likelihood of fluid contamination. This may be achieved through the use of rotating pinch rollers within the pump 122, which may rotate on a driveshaft to intermittently compress the tubing 116 against an anvil support.

Additionally or alternatively, pump 122 can include a linear peristaltic pump. With a linear peristaltic pump, surgical fluid 112 can be conveyed within the delivery tubing 116 by waves of contraction, directed externally onto the tubing 116, which are produced mechanically, typically by a series of compression fingers or pads which sequentially squeeze the tubing 116 against a support.

In some examples, the surgical fluid 112 may include saline, preferably normal (physiologic) saline, however, any other suitable electrically conductive fluids may be used instead or in addition. While a conductive fluid is preferred, surgical fluid 112 can also include a non-conductive (e.g., electrically insulative) fluid. The use of a non-conductive fluid is less preferred than a conductive fluid, however, the use of a non-conductive fluid still provides certain advantages over the use of dry electrodes including, for example, reduced occurrence of tissue adhering to electrodes of handheld device 106 and cooling of the electrodes and/or tissue. Therefore, it is also within the scope of the present disclosure to include the use of a non-conducting fluid, such as deionized water.

In embodiments, suction tubing 142 may flow through pump 122 to suction remnants (e.g., excess fluid, blood, tissue, byproducts, etc.) into the multifunctional electrosurgical device 130 at a treatment area (not shown) during operation of the multifunctional electrosurgical device 130 and control flow. In embodiments, the remnants may flow through the suction tubing 142, through pump 122 and into a waste container/receptacle 140.

In embodiments, the example electrosurgical unit 110 may selectively provide at least one or more monopolar RF power outputs and at least one or more bipolar power outputs, or both to a specified electrosurgical instrument, such as the multifunctional electrosurgical device 130. In one example, the electrosurgical unit 110 can be used for delivery of RF energy to instruments indicated for cutting and coagulation of soft tissue and for delivery of RF energy concurrent with fluid to instruments indicated for hemostatic sealing and coagulation of soft tissue and bone.

The features of electrosurgical unit 110 described are for illustration, and the electrosurgical units suitable for use with the multifunctional electrosurgical device 130 may include some, all, or other features than those described below.

In embodiments, the electrosurgical unit 110 is capable of operating in at least monopolar mode as well as multiple functions within the monopolar mode such as a monopolar cutting function, a monopolar coagulation function, and monopolar hemostasis, tissue scaling function, fluid irrigation, and suction, utilizing a single, retractable monopolar electrode at the distal tip of the multifunctional electrosurgical device 130. The electrosurgical unit 110 is operably configured to provide sufficient monopolar energy, e.g., RF energy, is provided to the multifunctional electrosurgical device 130 for each desired function. For example, a relatively low voltage or a continuous current may be provided for a cut function, a relatively higher voltage with a pulsed current may be provided, or an alternative power voltage and current may be provided, along with fluid, for hemostasis or tissue sealing, etc. to the multifunctional electrosurgical device 130 via the electrosurgical unit 110.

In embodiments, the electrosurgical unit 110 is capable of operating in at least bipolar mode as well as multiple functions within the bipolar mode, similar to that of the monopolar mode, however, utilizing at least in part, an active electrode and a return electrode at the distal tip of the multifunctional electrosurgical device 130. The electrosurgical unit 110 is operably configured to provide sufficient power, e.g., alternating-current (AC), created between the two electrical poles sufficient to realize the desired effect on the targeted area.

It is to be understood, while a monopolar mode and a bipolar mode have been depicted separately, the electrosurgical unit 110 is configured to output both monopolar power, bipolar power, irrigation, and suction individually and simultaneously to the multifunctional electrosurgical device 130.

In embodiments, the electrosurgical unit 110 provides RF energy to the active electrode(s) as a signal having a frequency in the range of 100 KHz to 10 MHz. Typically, this energy is applied in the form of bursts of pulses. Each burst typically has a duration in the range of 10 microseconds to 1 millisecond. The individual pulses in each burst typically each have a duration of 0.1 to 10 microseconds with an interval between pulses of 0.1 to 10 microseconds. The actual pulses are often sinusoidal or square waves and bi-phasic, that is alternating positive and negative amplitudes.

The electrical surgical unit 110 may include a power switch to turn the unit on and off and an RF power setting display to display the RF power supplied to the multifunctional electrosurgical device 130. The power setting display can display the RF power setting numerically in a selected unit such as watts.

In embodiments, electrosurgical unit 110 may include an RF power selector comprising RF power setting switches that are used to select or adjust the RF power setting. A user can push one power setting switch to increase the RF power setting and push the other power setting switch to decrease the RF power setting. In one example, power setting switches are membrane switches, soft keys, or as part of a touchscreen. In another example, the electrosurgical unit may include more than one power selectors such as a power selector corresponding with each of the different monopolar settings used in the different functions.

In embodiments, electrosurgical unit 110 may also include a fluid flow rate setting display and flow rate setting selector. The display can include indicator lights, and the flow rate selector can include switches. Pushing one of the flow rate switches selects a fluid flow rate, which is then indicated in display.

In embodiments, the relationship between the variables of fluid flow rate Q (such as in units of cubic centimeters per minute (cc/min)) and RF power setting Ps (such as in units of watts) can be configured to inhibit undesired effects such as tissue desiccation, electrode sticking, smoke production, char formation, and other effects while not providing a fluid flow rate Q at a corresponding RF power setting Ps not so great as to disperse too much electricity and or overly cool the tissue at the electrode/tissue interface. In embodiments, the electrosurgical unit 110 may be configured to increase the fluid flow rate Q generally linearly with an increasing RF power setting Ps for each of the fluid flow rate settings (e.g., low, medium, and high).

In embodiments, the electrosurgical unit 110 may be configured to include control of the pump 122. In embodiments, the speed of the pump 122, fluid irrigation, and fluid suction may be predetermined based on input variables such as the RF power setting and the fluid flow rate setting. In one example, the pump 122 can be integrated with the electrosurgical unit 110.

In embodiments, the multifunctional electrosurgical device 130 may be connected to the electrosurgical unit 110 via cable 126, which may include a plurality of electrically insulated wire conductors. The cable 126 may include plugs 134 that connect with receptacles 136 on the electrosurgical unit 110. In embodiments, a receptacle may correspond with an active electrode receptacle and one or more receptacles can correspond with controls on the multifunctional electrosurgical device 130. In embodiments, a receptacle can correspond with a second active electrode receptacle. An additional cable may connect a ground pad electrode to a ground pad receptacle of the electrosurgical unit 110. The fluid-delivery tubing 116 and the suction tubing 142 can be integrated with cable 126 and produced with the electrically insulated wires via plastic co-extrusion. In accordance, the multifunctional electrosurgical device 130 can be configured to both irrigate fluid 112 into a target treatment site, and subsequently aspirate residual remnants from the target treatment site without or with minimal interruption of an electrosurgical procedure, such as activating and deactivating a retractable monopolar electrode (e.g., for dissection), activating and deactivating bipolar electrodes, or a combination thereof.

For example, the multifunctional electrosurgical device 130 may be fluidically coupled, via suction tubing 142, to a suction source (e.g., pump 122 configured for simultaneous suction and irrigation or an external suction source) and a discharge reservoir 140. The multifunctional electrosurgical device 130 can include one or more user-input mechanisms, such as buttons, switches, levers, triggers, toggles, knobs, or the like 138, 139 and 140, configured to control irrigation and aspiration of surgical fluid 112, extraction and retraction of the monopolar electrode, activation and deactivation of the monopolar, activation and deactivation of the bipolar electrodes, etc. of the multifunctional electrosurgical device 130, as described herein.

While the multifunctional electrosurgical device 130 is described with reference to the electrosurgical unit 110 and other elements of system 100, it should be understood the description of the combination is for the purposes of illustrating system 100.

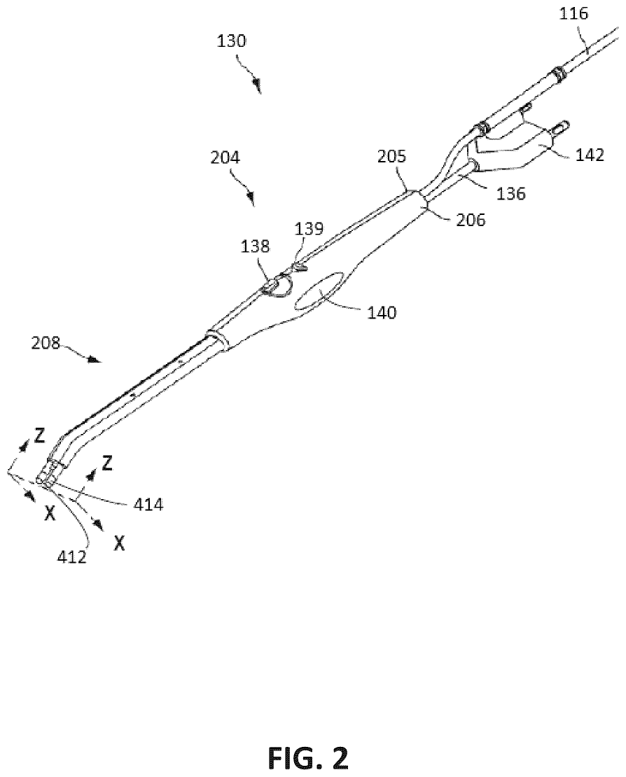

FIG. 2 is a perspective view of a multifunctional electrosurgical device 130, according to embodiments, that may be used in conjunction with electrosurgical unit 110 for selective monopolar electrosurgery, bipolar electrosurgery, fluid irrigation, and suction. The exemplary multifunctional electrosurgical device 130 includes an exemplary proximal handle 204 having mating lateral handle portions 205 and 206. The exemplary handle 204 is preferably made of a sterilizable, rigid, non-conductive material, such as a polymer (e.g., polycarbonate). Also, handle 204 is preferably configured slender, along with the rest of the multifunctional electrosurgical device 130, to facilitate a user of the multifunctional electrosurgical device 130 to hold and manipulate the multifunctional electrosurgical device 130 (e.g., in a manner similar to a writing utensil). The multifunctional electrosurgical device 130 may also include an electrical cable 136 which is connectable to electrosurgical unit 110, irrigation tubing 116 which is connectable to fluid source 120, and suction tubing 142 which is connectable to waste container/receptacle 140 (FIG. 1), which respectively provides radio-frequency energy, surgical fluid 112 to electrodes/targeted surgical area, and the removal of remnants near the electrodes/targeted surgical area.

Retained at and connected to or otherwise extruding from the distal end of shaft 208 are two laterally and spatially separated (by temporary empty space) contact elements comprising bipolar electrodes 412 and 414. In embodiments, the electrodes 412 and 414 may be configured as mirror images in size and shape, and may have a distal end with a surface devoid of edges (to provide a uniform current density) to treat tissue without cutting. Electrodes 412 and 414 may be formed from an electrically conductive metal, such as stainless steel, titanium, gold, silver, and/or platinum.

In embodiments, retained at, coupled to, and within, the distal end of the shaft 208 may also be a retractable, monopolar electrode in between or among the laterally and spatially separated bipolar electrodes 412 and 414. The retractable, monopolar electrode operably configured to selectively extrude from within the shaft 208 and retract within the shaft 208. In embodiments, the retractable, monopolar electrode is operably designed to extrude a distance beyond the distal edges of the laterally and spatially separated bipolar electrodes 412 and 414, such that the bipolar electrodes 412 and 414 do not operably interfere with the procedures and functionality of using the monopolar electrode.

In an embodiment, the longitudinal axes “Z” of electrodes 412 and 414 may be separated center-to-center (“CC”). As a result, when electrodes 412 and 414 may have a diameter ‘d’ mm and a spatial gap separation of ‘x’ mm between electrodes 412 and 414. the electrodes 412 and 414 are preferably configured to slide across a surface of a target tissue in the presence of the radio-frequency energy from electrosurgical unit 110. Fluid 112 may be selectively activated and irrigated from the fluid source 120 through the multifunctional electrosurgical device 130 and out the distal end. In embodiments, remnants may be suctioned from the targeted area through the multifunctional electrosurgical device 130 through inlets 432 and 434 integrated with electrodes 412 and 414, which may be in fluid connection with suction tubes 142. In embodiments, the electrodes 412 and 414 may each have a domed distal shape which may provide a smooth, blunt contour outer surface which is not pointed or sharp. In embodiments, in-between or otherwise within the spatial gap of the electrodes 412 and 414 may include a selectively activated and retractable monopolar electrode which may be extendable beyond the length of the length of the electrodes 412 and 414. It is to be understood that, while the distal tip of the shaft is depicted to be angled, other alternative shapes and angles are contemplated, such as distal portions being straight or linear relative to the shaft 208.

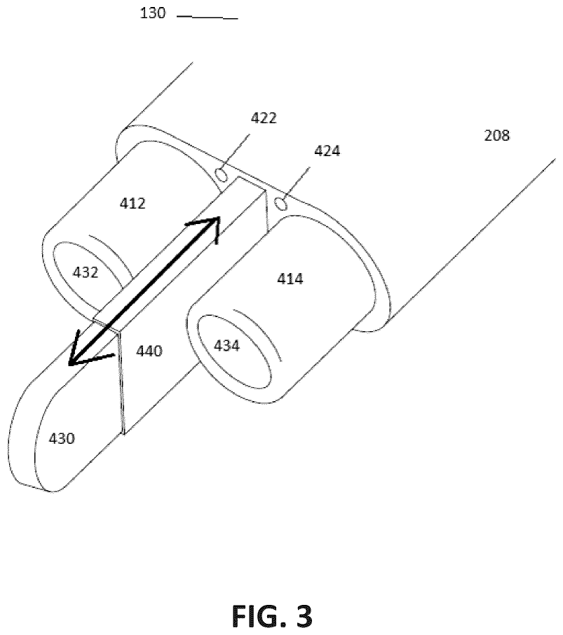

FIG. 3 illustrates an example embodiment of a distal end of a multifunctional electrosurgical device 130, including a retractable monopolar electrode 430 and bipolar electrodes 412 and 414. In embodiments, the bipolar electrodes 412 and 414 may be retained at, connected to, or otherwise coupled to the shaft 208. In embodiments, the shaft 208 may be of an elongated or stretched oval shape. In embodiments, the bipolar electrodes 412 and 414 may be laterally and spatially separated, such that the bipolar electrodes 412 and 414 may be coupled to each respective side of the distal portion of the elongated or stretched oval designed staff 208. In embodiments, the bipolar electrodes 412 and 414 may protrude outward, distally, from the distal portion of the shaft 208.

In embodiments, the bipolar electrodes 412 and 414 and the retractable monopolar electrode 430 are connected to electrosurgical unit 110 to provide RF power. The bipolar electrodes 412 and 414 may be configured to form an alternating-current electrical field in tissue located between electrodes 412 and 414. In the presence of alternating current, the electrodes 412 and 414 may alternate polarity between positive and negative charges with current flow from the positive to negative charge. In embodiments, heating of the tissue is performed by electrical resistance heating. In embodiments, the monopolar electrode 430 may be selectively extracted and activated, wherein current from the electrical surgical unit 110 passes current to the electrode 430, through the tissue and through a patient return pad. In embodiments, heating of the tissue is performed by electrical resistance heating.

In embodiments, the retractable monopolar electrode 430 may be housed or retained within the shaft 208 of the multifunctional electrosurgical device 130. The retractable monopolar electrode 430 may be selectively extendable, beyond the distance of the bipolar electrodes 412 and 414, such that monopolar electrosurgical procedures may be conducted at the targeted tissue area without interference from the bipolar electrodes 412 and 414. In embodiments, the monopolar electrode 430 may also include an insulation portion 440, which may also be selectively retractable with the monopolar electrode 430. For example, when the monopolar device 430 is activated/extracted by a user, via one or more of the user input mechanisms 138, 139 or 140, the monopolar electrode 430 may be extruded out of the shaft 208 and extend beyond the bipolar electrodes 412 and 414. Upon extension, the insulation portion 440 may also be extended such that the insulation portion encompasses the monopolar electrode, at least, the length of which the bipolar electrodes extend from the distal end of the shaft 208. In embodiments, when the user has completed their task/procedure with the monopolar electrode 430, the user may actuate one or more of the user input mechanisms 138, 139 or 140, to retract the monopolar electrode 430, including the insulation portion 440, back into the shaft 208.

In embodiments, one or more irrigation outlets 422 and 424 may be integrated within and through the multifunctional electrosurgical device 130 to facilitate fluid 112 onto the target tissue area during use, through the shaft 208. In embodiments, the irrigation outlets 422 and 424 may be configured to be positioned in between the laterally and spatially separated bipolar electrodes 412 and 414. Control of the irrigation flow may be controlled by one or more of the user input mechanisms 138, 139, or 140. Thus, during use of multifunctional electrosurgical device 130, fluid 112 from fluid source 120 may be communicated through one or more lumens of irrigation tubing 116, after which it flows through the lumens where it thereafter exits the multifunctional electrosurgical device 130 at the distal end from irrigation outlets 422 and 424 around electrodes 412 and 414 and the target tissue are.

Additionally, in embodiments, bipolar electrodes 412 and 414 may define respective inner lumens or inlets 432 and 434, which may double as integrated suction tubes 142 for suctioning remnants at the target tissue site. The bipolar electrodes 412 and 414 of distal portion may form substantially cylindrical, tubular shapes such that distal-most ends of electrodes 412 and 414 respectively define suction ports 432 and 434, each suction port 432 and 434 defining distal-most end of a central aspiration lumen running proximally through each electrode 412 and 414. Suction ports 432 and 434 are each an example of suctioning functions described in FIG. 1, in that they are configured to suction residual remnants from the target treatment site.

In embodiments, bipolar electrodes 412 and 414 and the monopolar electrode 430 may be selectively used individually or simultaneously. In embodiments, one or more user input mechanisms 138, 139 or 140 may be selectively configured to activate, deactivate, extend, retract, etc. the one or more electrodes. In embodiments, one or more user input mechanisms 138, 139, or 140 may be configured to selectively irrigate fluid or suction remnants, either separately or simultaneously, and/or function in combination with use of the electrodes 412, 414 and 430.

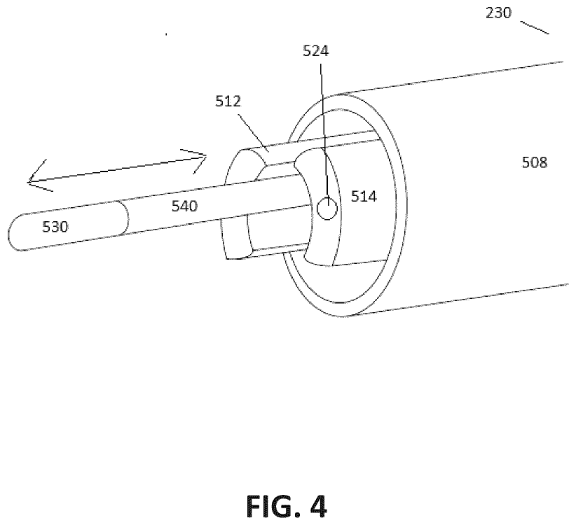

FIG. 4 illustrates an example, alternative embodiment of a distal end of a multifunctional electrosurgical device 230, including a retractable monopolar electrode 530 and bipolar electrodes 512 and 514. In embodiments, the shaft 508 and distal end may be of an alternative shape and design. For example, the multifunctional electrosurgical device may include a cylindrical shaft 508 and distal end of a cylindrical/round design, as depicted. In embodiments, the multifunctional electrosurgical device 230 may be constructed of a molded hollow, cylindrical shaft 508. In embodiments, the shaft may include a selectively retractable monopolar electrode 530 and bipolar electrodes 512 and 514 protruding from the distal portion of the shaft 508 of the multifunctional electrosurgical device. In embodiments, the bipolar electrodes 512 and 514 may be retained at, connected to, or otherwise coupled to the distal portion of the shaft 508. In embodiments, the bipolar electrodes 512 and 514 may be laterally and spatially separated. In embodiments, the bipolar electrodes 512 and 514 may be coupled at each respective edge of the distal portion of the cylindrical designed shaft 508.

In embodiments, the bipolar electrodes 512 and 514 and the retractable monopolar electrode 530 are connected to electrosurgical unit 110 to provide RF power. The bipolar electrodes 512 and 514 may be configured to form an alternating-current electrical field in tissue located between electrodes 512 and 514. In the presence of alternating current, the electrodes 512 and 514 may alternate polarity between positive and negative charges with current flow from the positive to negative charge. In embodiments, heating of the tissue is performed by electrical resistance heating. In embodiments, the retractable monopolar electrode 530 may be selectively extracted and activated, wherein current from the electrical surgical unit 110 passes current to the electrode 530, through the tissue and through a patient return pad. In embodiments, heating of the tissue is performed by electrical resistance heating.

In embodiments, the retractable monopolar electrode 530 may be housed or retained within the shaft 508 of the multifunctional electrosurgical device 230. The retractable monopolar electrode 530 may be selectively extendable, beyond the distance of the bipolar electrodes 512 and 514, such that monopolar electrosurgical procedures may be conducted at the targeted tissue area without interference from the bipolar electrodes 512 and 514. In embodiments, the monopolar electrode 530 may also include an insulation portion 540, which may also be selectively retractable with the monopolar electrode 530. For example, when the monopolar device 530 is activated/extracted by a user, via one or more of the user input mechanisms 138, 139 or 140, the monopolar electrode 530 may be extruded out of the shaft 508 and extend beyond the bipolar electrodes 512 and 514. In embodiments, the insulation portion 540 may include utilizing a heatshrink. Upon extension, the insulation portion 540 may also be extended such that the insulation portion 540 encompasses the monopolar electrode 530, at least, the length of which the bipolar electrodes 512 and 514 extend from the distal end of the shaft 508. In embodiments, when the user has completed their task/procedure with the monopolar electrode 530, the user may actuate one or more of the user input mechanisms 138, 139 or 140 to retract the monopolar electrode 530, including the insulation portion 540, back into the shaft 508.

In embodiments, one or more irrigation outlets 522 and 524 may be integrated within and through the multifunctional electrosurgical device 230 to facilitate fluid 112, via one or more irrigation tubes 116, onto the target tissue area during use. In embodiments, the one or more irrigation tubes 116 may be configured to flow through the bipolar electrodes 512 and 514, with the irrigation tubes ending at irrigation outlets 522 and 524. Control of the irrigation flow may be controlled by one or more of the user input mechanisms 138, 139, or 140. Thus, during use of multifunctional electrosurgical device 230, fluid 112 from fluid source 120 may be communicated through one or more lumens of irrigation tubing 116, after which it flows through the lumens where it thereafter exits the multifunctional electrosurgical device 130 at the distal end from irrigation outlets 522 and 524 around electrodes 412 and 414 and the target tissue are. It is to be understood that, while irrigation outlet 522 may not be depicted in FIG. 4, in embodiments, there may be a mirrored relationship between bipolar electrode 524, including irrigation outlet 524, and bipolar electrode 512, which includes the same or similar irrigation outlet (with a same or similar irrigation tubing flowing within) for fluid 112 irrigation.

Additionally, in embodiments, bipolar electrodes 512 and 514 at the distal portion of the multifunctional electrosurgical device 230 may form cylindrical, partial moon-like shapes (e.g., having at least a rounded or curved structure). In embodiments, the partial moon-like shape may be configured to more effectively contour to fit into or couple with the cylindrical shape of the shaft 508. In embodiments, the bipolar electrodes 512 and 514 may be press-in (or snap-in) electrodes, such that bipolar electrodes 512 and 514 may be interchangeable or otherwise easily integrated into the shaft 508. In embodiments, the press-in bipolar electrodes 512 and 514 do not run the full length of the shaft 508 to be electrically coupled to the electrosurgical unit 110. The press-in bipolar electrodes 512 and 514, while protruding a length from the distal portion of the shaft 508, may include a portion of the press-in electrodes 512 and 514 coupled within the distal portion of the shaft 508. In embodiments, one or more suction ports may be integrated within the shaft 508, via suction tubing 142, and be integrated to suction remnants at the target area, via the distal end of the shaft. In embodiments, the one or more suction ports may be incorporated within the bipolar electrodes 512 and 514. In embodiments, the suction tubing 142 and one or more suction ports 532 may be integrated in locations other than the bipolar electrodes 512 and 514, such as below or above the retractable monopolar electrode 530. in that they are configured to suction residual remnants from the target treatment site.

In embodiments, bipolar electrodes 512 and 514 and the monopolar electrode 530 may be selectively used individually or simultaneously. In embodiments, one or more user input mechanisms 138, 139 or 140 may be selectively configured to activated, deactivate, extend, retract, etc. the one or more electrodes. In embodiments, one or more user input mechanisms 138, 139, or 140 may be configured to selectively irrigate fluid or suction remnants, either separately or simultaneously, and/or function in combination with use of the electrodes 512, 514 and 530.

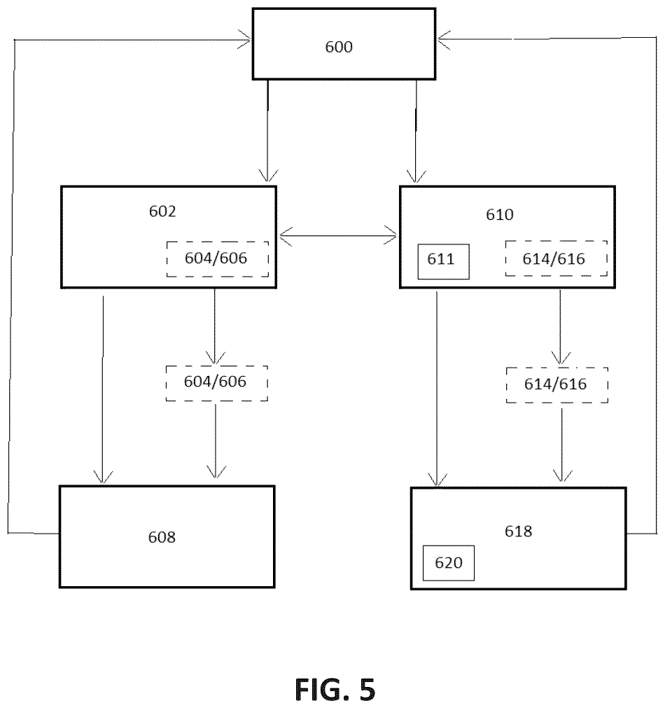

FIG. 5 illustrates an example block diagram method for use of a multifunctional electrosurgical device, according to embodiments. At 600, a multifunctional electrosurgical device may be available for use. At 602, the first and second bipolar electrodes may be activated, base preferences of a clinician and/or necessary circumstances arising during a surgical procedure. In embodiments, the first bipolar electrode may be selectively activated for delivering bipolar current to a target treatment area. In embodiments, the second bipolar electrode may be activated for receiving a return electrical current from the target treatment area and the first bipolar electrode. At 604 irrigation may be provided, via one or more irrigation tubing, to the treatment area. In embodiments, irrigation may be provided either during bipolar electrode activation, periodically between activations, or a combination thereof. At 606 option suction may be provided, either by an external suction device or by the optional one or more suction tubing, to remove remnants from the treatment area. In embodiments, suction may be provided either during bipolar electrode activation, periodically between activations, or a combination thereof. At 608, the first and second bipolar electrodes may be deactivated and await reactivation.

At 610, the retractable monopolar electrode may be activated, based preferences of a clinician and/or necessary circumstances arising during a surgical procedure. In embodiments, the retractable monopolar electrode may be house within the shaft of the multifunctional electrosurgical device. In embodiments, the retractable monopolar electrode may selectively be activated for delivering monopolar current to a target treatment area. In embodiments, the retractable monopolar electrode may be activated such that the retractable monopolar electrode extends outward the distal portion of the shaft. In embodiments, the retractable monopolar electrode may be extendable beyond the length of the protruding first and second bipolar electrodes. In embodiments, the retractable monopolar electrode may be at least partially surrounded by selectively extendable and retractable insulation material. The insulation material protecting against interference (e.g., physical or electrical) between the first and second bipolar electrodes and the retractable monopolar electrode. At 612, irrigation may be provided, via one or more irrigation tubing, to the treatment area. In embodiments, the one or more irrigation tubing may be integrated within the first or second bipolar electrodes. In embodiments, irrigation may be provided either during bipolar electrode activation, periodically between activations, or a combination thereof. At 616 optional suction may be provided, either by an external suction device or the optional one or more suction tubing, to remove remnants from the treatment area. In embodiments, suction may be provided either during bipolar electrode activation, periodically between activations, or a combination thereof. At 618, the first and second bipolar electrodes may be deactivated, wherein the electrical current may be deactivated. At 620, upon deactivation, the retractable monopolar electrode may be retracted into the shaft of the multifunctional electrosurgical device and await reactivation.

At 610, the retractable monopolar electrode may be activated, based preferences of a clinician At 622, while the first and second bipolar electrodes and retractable monopolar electrode has been described as being used individually, the first and second bipolar electrodes and retractable monopolar electrode may be configured to be activated individually or simultaneously, such that the first and second bipolar electrodes and the retractable monopolar electrode may be used simultaneously. In embodiments, at 624 the first and second bipolar electrodes and the retractable monopolar electrode may configure to be deactivated individually or simultaneously.

In examples, a multifunctional electrosurgical device comprising a proximal portion comprising at least one of an electrical connector coupled to an electrical surgical unit, one or more irrigation tubes coupled to a fluid source and one or more suction tubes coupled to a waste receptacle. An elongated, oval shaft including a distal portion comprising a first bipolar electrode extending distally from the distal portion, the first electrode configured to selectively provide a delivered electrical current to a target treatment area and a second bipolar electrode extending distally from the distal portion, the second electrode configured to receive a return electrical current from the target treatment area, wherein the first and second electrodes are laterally and spatially separated. The first and second bipolar electrodes integrated with the one or more suction tubes. The distal portion further comprising a retractable monopolar electrode, positioned in between the first and second electrode, selectively extendable from the distal portion and configured to selectively provide a delivered electrical current to the target treatment area. A handle portion in-between the proximal portion and distal portion, including one or more user input mechanisms for control of at least one of the bipolar or monopolar electrode activation, extraction and retraction of the retractable monopolar electrode, irrigation of a fluid, and suction at the treatment area.

In examples, suction of any remnants is provided and removed from the treatment area from the distal portion at one or more suction ports through the one or more suction tubes.

In examples, the retractable monopolar electrode is selectively extendable beyond the length of the first and second bipolar electrodes and is selectively retracted within the elongated, oval shaft.

In examples, the retractable monopolar electrode is at least partially encased by an insulation portion, wherein the insulation portion is selectively extendable and retractable with the monopolar electrode.

In examples, the insulation portion extends at least to the length of the first and second bipolar electrode from the distal portion during use of the monopolar electrode and insulates the retractable monopolar electrode from the first and second bipolar electrodes.

In examples, the retractable monopolar electrode may be activated and deactivated independently of the first and second bipolar electrodes.

In examples, the retractable monopolar electrode may be activated and deactivate simultaneously with the first and second bipolar electrodes.

In examples, a multifunctional electrosurgical device comprising a proximal portion comprising at least one of an electrical connector coupled to an electrical surgical unit and one or more irrigation tubes coupled to a fluid source. A cylindrical shaft including a distal portion comprising a first bipolar electrode extending distally from the distal portion, the first electrode configured to selectively provide a delivered electrical current to a target treatment area and a second bipolar electrode extending distally from the distal portion, the second electrode configured to receive a return electrical current from the target treatment area, wherein the first and second electrodes are curved and contour to the sides of the distal portion of the cylindrical shaft. The first and second bipolar electrodes integrated with the one or more irrigation tubes. The distal portion further comprising a retractable monopolar electrode, positioned in between the first and second electrode, selectively extendable from the distal portion and configured to selectively provide a delivered electrical current to the target treatment area. A handle portion in-between the proximal portion and distal portion, including one or more user input mechanisms for control of at least one of the bipolar or monopolar electrode activation, extraction and retraction of the retractable monopolar electrode, irrigation of a fluid, and suction at the treatment area.

In examples, fluid is provided to the treatment area from the distal portion at one or more irrigation ports through the first and second bipolar electrodes via the one or more irrigation tubes.

In examples, the retractable monopolar electrode is selectively extendable beyond the length of the first and second bipolar electrodes and wherein the retractable monopolar electrode is selectively retracted within the cylindrical shaft.

In examples, the retractable monopolar electrode is at least partially encased by an insulation portion, wherein the insulation portion is selectively extendable and retractable with the monopolar electrode.

In examples, the insulation portion extends at least to the length of the first and second bipolar electrode from the distal portion during use of the monopolar electrode and insulates the retractable monopolar electrode from provided electrical current from the first or second bipolar electrodes.

In examples, the retractable monopolar electrode may be activated and deactivated independently of the first and second bipolar electrodes.

In examples, the retractable monopolar electrode may be activated and deactivate simultaneously with the first and second bipolar electrodes.

In examples, the first and second bipolar electrodes are press-in and detachable electrodes within the distal portion of the cylindrical shaft.

In examples, a method of performing electrosurgery with a multifunctional electrosurgical device, the method comprising selectively delivering, via activating a first bipolar electrode, a delivered current to a target treatment area. Selectively receiving, via activating a second bipolar electrode, a return electrical current from the target treatment area. Selectively delivering, via activating a retractable monopolar electrode, a delivered current to the treatment area, wherein the retractable monopolar is selectively extruded and retracted from within a distal portion of a shaft of the multifunctional electrosurgical device.

Selectively delivering, via at least one irrigation tubing, a fluid to the target treatment area and selectively suctioning, via at least one suction tubing, remnants and fluid from the target treatment area, wherein one or more user input mechanisms are configured to activate and deactivate each aspect of selectively activating and deactivating the bipolar electrodes, activating and deactivating the monopolar electrode, delivering fluid to the target treatment area and suctioning remnants and fluid from the target treatment area.

In examples, at least one of one or more irrigation ports defined within at least one of the first and second bipolar electrodes, wherein fluid is selectively irrigated to the target treatment area, or one or more suction intake ports defined within at least one of the first and second bipolar electrodes, wherein remnants and fluid is selectively suctioned from the target treatment area.

In examples, extruding the retractable monopolar electrode beyond the length of the first and second bipolar electrodes.

In examples, insulating at least a portion of the monopolar electrode with an encompassing, selectively retractable insulation material.

In examples, delivering current to the target treatment area via the first and second bipolar electrodes and the retractable monopolar electrode individually, wherein during current delivery of the first and second bipolar electrodes, the retractable monopolar electrode is selectively retracted into a shaft of the multifunctional electrosurgical device; and during current delivery of the retractable monopolar electrode, the first and second bipolar electrodes are deactivated.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

What is claimed is:

1. A multifunctional electrosurgical device comprising:
a proximal portion comprising at least one of an electrical connector coupled to an electrical surgical unit, one or more irrigation tubes coupled to a fluid source and one or more suction tubes coupled to a waste receptacle;
an elongated, oval shaft including a distal portion comprising a first bipolar electrode extending distally from the distal portion, the first electrode configured to selectively provide a delivered electrical current to a target treatment area and a second bipolar electrode extending distally from the distal portion, the second electrode configured to receive a return electrical current from the target treatment area, wherein the first and second electrodes are laterally and spatially separated;
the first and second bipolar electrodes integrated with the one or more suction tubes;
the distal portion further comprising a retractable monopolar electrode, positioned in between the first and second electrode, selectively extendable from the distal portion and configured to selectively provide a delivered electrical current to the target treatment area;
a handle portion in-between the proximal portion and distal portion, including one or more user input mechanisms for control of at least one of the bipolar or monopolar electrode activation, extraction and retraction of the retractable monopolar electrode, irrigation of a fluid, and suction at the treatment area.

2. The multifunctional electrosurgical device of claim 1, wherein suction of any remnants is provided and removed from the treatment area from the distal portion at one or more suction ports through the one or more suction tubes.

3. The multifunctional electrosurgical device of claim 1, wherein the retractable monopolar electrode is selectively extendable beyond the length of the first and second bipolar electrodes and is selectively retracted within the elongated, oval shaft.

4. The multifunctional electrosurgical device of claim 1, wherein the retractable monopolar electrode is at least partially encased by an insulation portion, wherein the insulation portion is selectively extendable and retractable with the monopolar electrode.

5. The multifunctional electrosurgical device of claim 4, wherein the insulation portion extends at least to the length of the first and second bipolar electrode from the distal portion during use of the monopolar electrode and insulates the retractable monopolar electrode from the first and second bipolar electrodes.

6. The multifunctional electrosurgical device of claim 1, wherein the retractable monopolar electrode may be activated and deactivated independently of the first and second bipolar electrodes.

7. The multifunctional electrosurgical device of claim 1, wherein the retractable monopolar electrode may be activated and deactivate simultaneously with the first and second bipolar electrodes.

8. A multifunctional electrosurgical device comprising:
a proximal portion comprising at least one of an electrical connector coupled to an electrical surgical unit and one or more irrigation tubes coupled to a fluid source;
a cylindrical shaft including a distal portion comprising a first bipolar electrode extending distally from the distal portion, the first electrode configured to selectively provide a delivered electrical current to a target treatment area and a second bipolar electrode extending distally from the distal portion, the second electrode configured to receive a return electrical current from the target treatment area, wherein the first and second electrodes are curved and contour to the sides of the distal portion of the cylindrical shaft;
the first and second bipolar electrodes integrated with the one or more irrigation tubes;
the distal portion further comprising a retractable monopolar electrode, positioned in between the first and second electrode, selectively extendable from the distal portion and configured to selectively provide a delivered electrical current to the target treatment area;
a handle portion in-between the proximal portion and distal portion, including one or more user input mechanisms for control of at least one of the bipolar or monopolar electrode activation, extraction and retraction of the retractable monopolar electrode, irrigation of a fluid, and suction at the treatment area.

9. The multifunctional electrosurgical device of claim 8, wherein fluid is provided to the treatment area from the distal portion at one or more irrigation ports through the first and second bipolar electrodes via the one or more irrigation tubes.

10. The multifunctional electrosurgical device of claim 8, wherein the retractable monopolar electrode is selectively extendable beyond the length of the first and second bipolar electrodes and wherein the retractable monopolar electrode is selectively retracted within the cylindrical shaft.

11. The multifunctional electrosurgical device of claim 8, wherein the retractable monopolar electrode is at least partially encased by an insulation portion, wherein the insulation portion is selectively extendable and retractable with the monopolar electrode.

12. The multifunctional electrosurgical device of claim 11, wherein the insulation portion extends at least to the length of the first and second bipolar electrode from the distal portion during use of the monopolar electrode and insulates the retractable monopolar electrode from provided electrical current from the first or second bipolar electrodes.

13. The multifunctional electrosurgical device of claim 8, wherein the retractable monopolar electrode may be activated and deactivated independently of the first and second bipolar electrodes.

14. The multifunctional electrosurgical device of claim 8, wherein the retractable monopolar electrode may be activated and deactivate simultaneously with the first and second bipolar electrodes.

15. The multifunctional electrosurgical device of claim 8, wherein the first and second bipolar electrodes are press-in and detachable electrodes within the distal portion of the cylindrical shaft.

16. A method of performing electrosurgery with a multifunctional electrosurgical device, the method comprising:
selectively delivering, via activating a first bipolar electrode, a delivered current to a target treatment area;
selectively receiving, via activating a second bipolar electrode, a return electrical current from the target treatment area;
selectively delivering, via activating a retractable monopolar electrode, a delivered current to the treatment area, wherein the retractable monopolar is selectively extruded and retracted from within a distal portion of a shaft of the multifunctional electrosurgical device;
selectively delivering, via at least one irrigation tubing, a fluid to the target treatment area; and
selectively suctioning, via at least one suction tubing, remnants and fluid from the target treatment area, wherein one or more user input mechanisms are configured to activate and deactivate each aspect of selectively activating and deactivating the bipolar electrodes, activating and deactivating the monopolar electrode, delivering fluid to the target treatment area and suctioning remnants and fluid from the target treatment area.

17. The method of performing electrosurgery with a multifunctional electrosurgical device of claim 16, further comprising at least one of one or more irrigation ports defined within at least one of the first and second bipolar electrodes, wherein fluid is selectively irrigated to the target treatment area, or one or more suction intake ports defined within at least one of the first and second bipolar electrodes, wherein remnants and fluid is selectively suctioned from the target treatment area.

18. The method of performing electrosurgery with a multifunctional electrosurgical device of claim 16, further comprising, extruding the retractable monopolar electrode beyond the length of the first and second bipolar electrodes.

19. The method of performing electrosurgery with a multifunctional electrosurgical device of claim 16, further comprising insulating at least a portion of the monopolar electrode with an encompassing, selectively retractable insulation material.

20. The method of performing electrosurgery with a multifunctional electrosurgical device of claim 16, further comprising:
delivering current to the target treatment area via the first and second bipolar electrodes and the retractable monopolar electrode individually, wherein during current delivery of the first and second bipolar electrodes, the retractable monopolar electrode is selectively retracted into a shaft of the multifunctional electrosurgical device; and
during current delivery of the retractable monopolar electrode, the first and second bipolar electrodes are deactivated.
